(19) United States
(12) Patent Application Publication
(10) Pub. No.: US 2016/0083742 A1
(43) Pub. Date: Mar. 24, 2016

(54) Modified Plants for Producing Aroma/Fine/Specialty Chemicals

(71) Applicant: WASHINGTON STATE UNIVERSITY, Pullman, WA (US)

(72) Inventors: Michael A. Costa, Pullman, WA (US); Norman G. Lewis, Pullman, WA (US); Laurence B. Davin, Pullman, WA (US)

(73) Assignee: Washington State University, Pullman, WA (US)

(21) Appl. No.: 14/783,745

(22) PCT Filed: Apr. 10, 2014
(86) PCT No.: PCT/US14/33628

§ 371 Date: Oct. 9, 2015

Related U.S. Application Data
(60) Provisional application No. 61/849,839, filed on Aug. 5, 2013.
(60) Provisional application No. 61/811,005, filed on Apr. 11, 2013.

(51) Int. Cl. C12N 15/82; C12P 19/44; C12P 7/22

(52) U.S. Cl. CPC C12N 15/8257; C12P 7/22; C12P 19/44

(57) ABSTRACT

The present disclosure describes genetically modified plants that contain one or more exogenous genes associated with aroma/fine/specialty compound biosynthesis, which are capable of producing aroma/fine/specialty compounds.

### CROSS-REFERENCE TO RELATED APPLICATION

This application claims the benefit under 35 U.S.C. §119(e) of U.S. Provisional Patent Application No. 61/811,005 filed Apr. 11, 2013, and U.S. Provisional Patent Application No. 61/849,839 filed Aug. 5, 2013 where these Provisional applications are incorporated herein by reference in its entirety.

### FEDERALLY SPONSORED RESEARCH

This disclosure was made with government support under Washington State University Agricultural Research Center Federal Special Grant Project No. WNP00768. The Government has certain rights in the invention.

### SEQUENCE LISTING

The Sequence Listing associated with this application is provided in text format in lieu of a paper copy, and is hereby incorporated by reference into the specification. The name of the text file containing the Sequence Listing is Sequence_listing.txt. The text file is about 3 KB, was created on Aug. 5, 2013, and is being submitted electronically via EFS-Web.

### BACKGROUND

Numerous commodity and specialty chemicals and other materials may be obtained from dwindling petroleum and coal sources. For example, several aromatics (e.g., styrenes and ethyl benzene) are synthetic products massively obtained annually as commodity chemicals from non-renewable and finite petrochemical resources. However, such synthetic procedures have considerable drawbacks that include the use of harsh synthetic conditions, production of undesirable side-products, and the use of non-renewable petrochemical inputs.

Some chemicals and materials may be isolated as highly valued and highly valuable natural products from plant resources. Using plants to produce chemicals does not have the drawbacks described above with respect to those produced from petrochemical sources. However, the isolation of chemicals from plants can be both much more expensive to produce and more commercially valuable than synthetic production of the same chemicals based on petrochemical inputs.

There are thus growing and urgent needs to increasingly obtain sustainable, renewable, domestic sources of commodity and specialty chemicals and other materials currently obtained from, for example, plant-based resources at scale and cost needed.

### SUMMARY

Embodiments of the present disclosure relate to a modified plant including one or more exogenous polynucleotides encoding one or more enzymes associated with aroma/fine/specialty compound biosynthesis.

Embodiments of the present disclosure also relate to a method for generating a modified plant. The method may include contacting one or more plant cells with one or more exogenous polynucleotides encoding one or more enzymes associated with aroma/fine/specialty compound biosynthesis, and cultivating the one or more plant cells to generate the modified plant.

Embodiments of the present disclosure also relate to a method for producing aroma/fine/specialty compounds from a modified plant. The method may include cultivating a modified plant to produce one or more aroma/fine/specialty compounds, wherein the modified plant includes one or more exogenous polynucleotides encoding one or more enzymes associated with biosynthesis of the aroma/fine/specialty compounds, and isolating the aroma/fine/specialty compounds.

In some embodiments of the methods and/or compositions of the present disclosure, the one or more enzymes may include a phenylacetaldehyde synthase (PAAS). In certain embodiments, the PAAS is a Rosa hybrida PAAS. In particular embodiments, the Rosa hybrida PAAS is Rosa hybrida cv. Fragrant Cloud phenylacetaldehyde synthase (RhFCPAAS), or a biologically active fragment or variant thereof. In certain embodiments, the PAAS is a Petunia hybrida PAAS. In particular embodiments, the Petunia hybrida PAAS is the Petunia hybrida cv. Mitchell phenylacetaldehyde synthase (PhMPAAS), or a biologically active fragment or variant thereof.

In some embodiments of the methods and/or compositions of the present disclosure, the one or more enzymes may include a phenylacetaldehyde reductase (PAR). In certain embodiments, the PAR is Lycopersicum esculentum phenylacetaldehyde reductase (LePAR1), or a biologically active fragment or variant thereof.

In some embodiments of the methods and/or compositions of the present disclosure, the one or more enzymes may include an acyltransferase. In certain embodiments, the acyltransferase is a Larrea tridentata acyltransferase (LtCAAT1), or a biologically active fragment or variant thereof.

In some embodiments of the methods and/or compositions of the present disclosure, the one or more enzymes may include a propenylphenol synthase. In certain embodiments, the propenylphenol synthase is a Larrea tridentata propenylphenol synthase (LtPPS1), or a biologically active fragment or variant thereof.

In some embodiments of the methods and/or compositions of the present disclosure, the one or more enzymes may include an allylphenol synthase. In certain embodiments, the allylphenol synthase is a Larrea tridentata allylphenol synthase (LtAPS1), or a biologically active fragment or variant thereof.

In some embodiments of the methods and/or compositions of the present disclosure, the one or more enzymes may include a PAAS and a PAR. In some embodiments of the methods and/or compositions of the present disclosure, the one or more enzymes may include an acyltransferase and a propenylphenol or allylphenol synthase. In certain embodiments of the methods and/or compositions of the present disclosure, the one or more enzymes may include a PAAS, a PAR, an acyltransferase, and a propenylphenol or allylphenol synthase.

In some embodiments of the methods and/or compositions of the present disclosure, the modified plant produces one or more aroma compounds. In certain embodiments, the one or more aroma compounds may include at least one or more metabolites associated with 2-phenylethanol synthesis. In certain embodiments, the one or more aroma compounds may include a 2-phenylethanol. In particular embodiments, accumulation of the 2-phenylethanol in parts of the modified plant is at least 0.025% fresh weight. In certain embodiments, the one or more aroma compounds may include a carbohydrate or other forms of covalent derivative associated with 2-phenylethanol. In particular embodiments, accumulation of the carbohydrate or the other forms of covalent derivative in the modified plant is at least 0.5% dry weight. In some instances, the derivative is 2-phenylethyl-glucoside. In certain embodiments, the modified plant produces an allyl phenol or a propenyl phenol. In certain embodiments, the modified plant produces at least one of a 2-phenylethanol, allyl phenol and a propenyl phenol.

In some embodiments of the methods and/or compositions of the present disclosure, the modified plant produces one or more aroma compounds that are sequestered in the modified plant. In certain embodiments, the one or more aroma compounds are sequestered as a covalent derivative associated with the one or more aroma compounds. In particular embodiments, the one or more aroma compounds are sequestered as a non-toxic derivative associated with the one or more aroma compounds. In particular embodiments, the one or more aroma compounds are sequestered via glycosylation or acylation. In particular embodiments, the one or more aroma compounds are sequestered as a carbohydrate derived from the one or more aroma compounds. In particular embodiments, the one or more aroma compounds include a 2-phenylethanol. For example, the 2-phenylethanol is sequestered as a 2-phenylethyl-glucoside and/or other ethylated forms. In particular embodiments, the one or more aroma compounds include an allyl phenol or a propenyl phenol.

In some embodiments of the methods and/or compositions of the present disclosure, the one or more exogenous polynucleotides are present in one or more expression constructs. In certain embodiments, the one or more expression constructs may include a promoter sequence to allow expression of the one or more enzymes in the modified plant. In some embodiments, the one or more expression constructs may include a terminator sequence.

In some embodiments of the methods and/or compositions of the present disclosure, the modified plant is a woody plant. In certain embodiments, the woody plant is a Populus or an Alnus species. In some embodiments, the modified plant is a hybrid poplar. In some embodiments, the modified plant may be a hybrid poplar, a Populus tremula×P. alba, a red alder, or an Alnus rubra.

In some embodiments of the method for producing aroma/fine/specialty compounds, the isolating the aroma/fine/specialty compounds may include extracting the aroma/fine/specialty compounds using an organic solvent such as an ether, or an aqueous miscible solvent such as an alcohol., or steam distillation, or enzymatic hydrolysis

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1 illustrates biosynthetic pathways leading to 2-phenylethanol production in plants.

FIG. 2 shows production of 2-phenylethanol in E. coli expressing RhFCPAAS and LePAR. 2-Phenylethanol production in E. coli transformed with an empty vector was not observed.

FIG. 3 shows 2-phenylethanol (A) and 2-phenylethyl glucoside (B) content in young leaves (302), old leaves (304) and stem (306) from Populus tremula×P. alba wild type, as well as Populus tremula×P. alba transformed with RhFCPAAS/LePAR1 or PhMPAAS/LePAR1 constructs.

FIG. 4 illustrates the comparison of RhFCPAAS, PtriPAAS, PhMPAAS amino acid sequences associated with conserved (pyridoxal 5′-phosphate)-binding residue.

FIG. 5 shows a partial UPLC-MS spectra of leaf extracts obtained from Populus tremula×P. alba wild type (WT, bottom curve), as well as from Populus tremula×P. alba transformed with RhFCPAAS/LePAR1 (top curve) or PhMPAAS/LePAR1 (middle curve) constructs showing presence of a peak at m/z 307.1152 corresponding to 2-phenylethyl glucoside.

FIG. 6 illustrates spatial distribution of 2-phenylethyl glucoside in a leaf of a transgenic poplar hybrid as analyzed by MALDI imaging mass spectrometry. MALDI image and ion intensity map of 2-phenylethyl glucoside (m/z 323.09, potassium adduct) on transgenic poplar hybrid leaf expressing RhFCPAAS/LePAR1 obtained with spatial resolution of 50 μm and using 2,5 dihydroxybenzoic acid as matrix.

FIG. 7 illustrates a biosynthetic pathway leading to allyl and propenyl phenols.

FIG. 8 shows allyl/propenyl phenol (and glucoside) content in young (gray) and old (dark) leaves from Populus tremula×P. alba wild type (WT) and transgenic lines expressing LtCAAT1/LtPPS1.

FIG. 9 shows partial UPLC-MS spectra of leaf extracts obtained from Populus tremula×P. alba wild type (WT, bottom curve), and from Populus tremula×P. alba transformed with LtCAAT1/LtPPS1 (top curve) constructs showing the presence of a peak at m/z 349.1258 corresponding to eugenol/isoeugenol glucosides.

FIG. 10 shows allyl phenol (and glucoside) content in young (gray) and old (dark) leaves from Populus tremula×P. alba wild type (WT) and transgenic lines expressing LtCAAT1/LtAPS1.

FIG. 11 shows partial UPLC-MS spectra of leaf extracts obtained from Populus tremula×P. alba wild type (WT, bottom curve), and from Populus tremula×P. alba transformed with LtCAAT1/LtAPS1 (top curve) constructs showing the presence of a peak at m/z 319.1152 corresponding to chavicol glucoside.

### DETAILED DESCRIPTION

### Overview

Embodiments of the present disclosure contemplate a use of the modified plants as a biotechnological platform for producing chemical compounds, such as aroma/fine/specialty chemicals. The present disclosure relates, in part, to the demonstration that woody plants, such as hybrid poplars, red alders, and etc., can be genetically modified to synthesize certain aroma/fine/specialty compounds such as 2-phenylethanol, eugenol, and etc. These woody plants do not naturally produce the certain aroma compounds in any significant amount, or the modified woody plants produce increased amounts of the certain aroma/fine/specialty compounds, as compared with wide-type woody plants. For example, as shown in the accompanying Examples, the addition of one or more polynucleotide sequences that encode one or more enzymes associated with aroma/fine/specialty compound synthesis renders the woody plants capable of converting their naturally-occurring intermediate molecules into aroma/fine/specialty compounds and/or derivatives of the aroma/fine/specialty compounds.

Examples of enzymes associated with the aroma/fine/specialty compound synthesis may include enzymes having a phenylacetaldehyde synthase (PAAS) activity and/or enzymes having a phenylacetaldehyde reductase (PAR) activity. Specifically, PAAS enzymes catalyze the production of phenylacetaldehyde molecules, an immediate pre-cursor to 2-phenylethanol, and PAR enzymes catalyze the production of 2-phenylethanol molecules from the phenylacetaldehyde molecules.

In addition, examples of enzymes associated with the aroma/fine/specialty compound synthesis may include enzymes having an acyltransferase activity and/or enzymes having an allyl/propenyl phenol synthase activity. Specifically, acyltransferase and propenylphenol synthase enzymes catalyze conversion from monolignols to propenyl phenols, and acyltransferase and allyl phenol synthase enzymes catalyze conversion from monolignols to allyl phenols.

### DEFINITIONS

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The term “biologically active fragment”, as applied to fragments of a reference polynucleotide or polypeptide sequence, refers to a fragment that has at least about 0.1, 0.5, 1, 2, 5, 10, 12, 14, 16, 18, 20, 22, 24, 26, 28, 30, 35, 40, 45, 50, 55, 60, 65, 70, 75, 80, 85, 90, 95, 96, 97, 98, 99, 100, 110, 120, 150, 200, 300, 400, 500, 600, 700, 800, 900, 1000% or more of the activity of a reference sequence. The term “reference sequence” refers generally to a nucleic acid coding sequence, or amino acid sequence, to which another sequence is being compared. All sequences provided in the Sequence Listing are also included as reference sequences.

The term “biologically active variant”, as applied to variants of a reference polynucleotide or polypeptide sequence, refers to a variant that has at least about 0.1, 0.5, 1, 2, 5, 10, 12, 14, 16, 18, 20, 22, 24, 26, 28, 30, 35, 40, 45, 50, 55, 60, 65, 70, 75, 80, 85, 90, 95, 96, 97, 98, 99, 100, 110, 120, 150, 200, 300, 400, 500, 600, 700, 800, 900, 1000% or more of the activity (e.g., an enzymatic activity) of a reference sequence. The term “reference sequence” refers generally to a nucleic acid coding sequence, or amino acid sequence, to which another sequence is being compared. The term “variant” encompasses biologically active variants, which may also be referred to as functional variants.

Included within the scope of the present disclosure are biologically active fragments of at least about 18, 19, 20, 21, 22, 23, 24, 25, 26, 27, 28, 29, 30, 40, 50, 60, 70, 80, 90, 100, 120, 140, 160, 180, 200, 220, 240, 260, 280, 300, 320, 340, 360, 380, 400, 500, 600 or more contiguous nucleotides or amino acid residues in length, including all integers in between, which comprise or encode a polypeptide having an activity of a reference polynucleotide or polypeptide. Representative biologically active fragments and variants generally participate in an interaction, e.g., an intra-molecular or an inter-molecular interaction. An inter-molecular interaction can be a specific binding interaction or an enzymatic interaction. Examples of enzymatic interactions or activities include, without limitation, phenylacetaldehyde synthase activity, phenylacetaldehyde reductase activity, acyltransferase activity, propenylphenol synthase activity, allylphenol synthase activity, glucosyl transferases and other carcohydrate transferases, shikimate/chorismate transferases, arogenate dehydratase and other enzymatic activities such as transporters of these metabolic products to specific subcellular, tissue or organ compartments.

By “coding sequence” is meant any nucleic acid sequence that contributes to the code for the polypeptide product of a gene. By contrast, the term “non-coding sequence” refers to any nucleic acid sequence that does not contribute to the code for the polypeptide product of a gene.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

By “corresponds to” or “corresponding to” is meant (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties or by post-translational modification techniques as would be understood in the art. The term “derivative” is also meant a chemical compounds that has been derived from the basic structure by modification via chemical reaction, for example, by conjugation or complexing with other chemical moieties (e.g., oxidation, decarboxylation, glycosylation, acylation, etc.). The term “derivative” also includes within its scope alterations that have been made to a parent sequence/structure including additions or deletions that provide for functionally equivalent molecules.

By “enzyme reactive conditions” it is meant that any necessary conditions are available in an environment (i.e., such factors as temperature, pH, lack of inhibiting substances) which will permit the enzyme to function. Enzyme reactive conditions can be either in vitro, such as in a test tube, or in vivo, such as within a cell.

As used herein, the terms “function” and “functional” and the like refer to a biological, enzymatic, or therapeutic function.

By “gene” is meant a unit of inheritance that occupies a specific locus on a chromosome and consists of transcriptional and/or translational regulatory sequences and/or a coding region and/or non-translated sequences (i.e., introns, 5′ and 3′ untranslated sequences).

“Homology” refers to the percentage number of amino acids that are identical or constitute conservative substitutions. Homology may be determined using sequence comparison programs such as GAP (Deveraux et al., 1984, Nucleic Acids Research 12, 387-395) which is incorporated herein by reference. In this way sequences of a similar or substantially different length to those cited herein could be compared by insertion of gaps into the alignment, such gaps being determined, for example, by the comparison algorithm used by GAP.

By “isolated” is meant material that is substantially or essentially free from components that normally accompany it in its native state. For example, an “isolated polynucleotide”, as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell.

The terms “modulating” and “altering” include “increasing” and “enhancing” as well as “decreasing” or “reducing,” typically in a statistically significant or a physiologically significant amount or degree relative to a control. By “increased” or “increasing” is included the ability of one or more modified plants, e.g., poplars, to produce (e.g., intracellularly accumulate and/or secrete and/or transport) a greater amount of one or more chemical compounds, relative to a control plant, typically of the same species, such as an unmodified (wild-type) plant. Examples of the chemical compounds are described herein.

An “increased” or “enhanced” amount is typically a “statistically significant” amount, and may include an increase that is 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

Production of chemical compounds, such as 2-phenylethanol, eugenol, and related derivatives thereof, can be measured according to techniques known in the art, and techniques described herein. Production of 2-phenylethanol can be measured, for example, UPLC-MS analysis, MALDI imaging, and etc. For example, certain modified plants described herein may produce at least about 0.01%, 0.02%, 0.03%, 0.04%, 0.05%, 0.06%, 0.07%, 0.08%, 0.09%, 0.10% in fresh weight for 2-phenylethanol in leaves of a hybrid poplar; and/or in the range of at least about 0.01%-0.02%, 0.02%-0.03%, 0.03%-0.04%, 0.04%-0.05%, 0.05%-0.06%, 0.06%-0.07%, 0.07%-0.08%, 0.08%-0.09%, 0.09%-0.10% in fresh weight for 2-phenylethanol in leaves of a hybrid poplar. For example, certain modified plants described herein may produce at least about 0.1%, 0.2%, 0.4%, 0.6%, 0.8%, 1%, 1.2%, 1.4%, 1.6%, 1.8%, 2% in dry weight for derivative 2-phenylethyl-glucoside in leaves of a hybrid poplar; and/or in the range of at least about 0.1%-0.2%, 0.2%-0.4%, 0.4%-0.6%, 0.6%-0.8%, 0.8%-16%, 1%-1.2%, 1.2%-1.4%, 1.4%-1.6%, 1.6%-1.8%, 1.8%-2% in dry weight for derivative 2-phenylethyl-glucoside in leaves of a hybrid poplar. Production of eugenol, and other aroma/fine/specialty compounds, can be measured similarly.

A “decreased” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

As used herein, the term “fine compounds” refers to complex, single, pure chemical substances. For example, fine chemicals may be produced in limited quantities in multipurpose plants by multistep batch chemical or biotechnological processes and may be used as starting materials for specialty compound for, for example, pharmaceuticals, biopharmaceuticals, agrochemicals, and etc.

As used herein, the term “aroma compounds” refers to volatile or non-volatile compounds that are perceived by receptor sites of perceptive organs, for example odor receptors, smell receptors, and etc. Examples of “aroma compounds” include specialty chemicals and associated precursors such as esters (e.g., Geranyl acetate, Methyl formate, Methyl acetate, Methyl propionate, Methyl propanoate, Methyl butyrate, Methyl butanoate, Ethyl acetate, Ethyl butyrate, Ethyl butanoate, Isoamyl acetate, Pentyl butyrate, Pentyl butanoate, Pentyl pentanoate, Octyl acetate, Benzyl acetate, Methyl anthranilate, and etc), Linear terpenes (e.g., Myrcene, Geraniol, Nerol, Citral, lemonal, Geranial, neral, Citronellal, Citronellol, Linalool, Nerolidol, and etc), Cyclic terpenes (e.g., Limonene, Camphor, Terpineol, alpha-lonone, Thujone, and etc), Aromatic (e.g., Benzaldehyde, Eugenol, Cinnamaldehyde, Ethyl maltol, Vanillin, Anisole, Anethole, Estragole, Thymol, and etc), Amines (e.g., Trimethylamine, Putrescine, Diaminobutane, Cadaverine, Pyridine, Indole, Skatole, and etc), alcohols (e.g., Furaneol, 1-Hexanol, cis-3-Hexen-1-ol, Menthol, 2-phenylethanol, and etc), and etc.

Additional examples of aroma/fine/specialty compounds and associated precursors include Acetaldehyde, Hexanal, cis-3-Hexenal, Furfural, Hexyl cinnamaldehyde, Isovaleraldehyde, Anisic aldehyde, Cuminaldehyde, Fructone, Hexyl acetate, Ethyl methylphenylglycidate, Cyclopentadecanone, Dihydrojasmone, Oct-1-en-3-one, 2-Acetyl-1-pyrroline, 6-Acetyl-2,3,4,5-tetrahydropyridine, gamma-Decalactone, gamma-Nonalactone, delta-Octalactone, Jasmine lactone, Massoia lactone, Wine lactone, Sotolon, allyl thiol, methanethiol, Ethanethiol, 2-Methyl-2-propanethiol, Butane-1-thiol, Grapefruit mercaptan, Methanethiol, Furan-2-ylmethanethiol, Benzyl mercaptan, Methylphosphine, dimethylphosphine, Phosphine, Diacetyl, Acetoin, Nerolin, Tetrahydrothiophene, 2,4,6-Trichloroanisole, pyrazine, and the like.

As used herein, the term “2-phenylethanol,” refers to an organic compound with the formula C₆H₅CH₂CH₂OH.

As used herein, the term “eugenol,” refers to a phenylpropene, an allyl chain-substituted guaiacol. Examples of “eugenol” include propenyl phenol, allyl phenol, and etc.

As used herein, the term “propenyl phenol,” refers to eugenol/isoeugenol and similar structures bearing the same or similar core.

As used herein, the term “Allyl phenol,” refers to eugenol/isoeugenol and similar structures bearing the same or similar core.

By “obtained from” is meant that a sample, for example, a polynucleotide, polypeptide or chemical compound, is isolated from, or derived from, a particular source, such as a desired organism or a specific tissue within a desired organism. “Obtained from” can also refer to the situation in which a polynucleotide or polypeptide sequence is isolated from, or derived from, a particular organism or tissue within an organism. For example, a polynucleotide sequence encoding a reference polypeptide described herein may be isolated from a variety of prokaryotic or eukaryotic organisms, or from particular tissues or cells within certain eukaryotic organism. In addition, “Obtained from” can also refer to the situation in which a chemical compounds is produced by, extracted from, isolated from, and/or derived from, a particular organism or tissue within an organism, such as plants.

The term “operably linked” as used herein means placing a gene under the regulatory control of a promoter, which then controls the transcription and optionally the translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e. the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting; i.e., the gene from which it is derived. “Constitutive promoters” are typically active, i.e., promote transcription, under most conditions. “Inducible promoters” are typically active only under certain conditions, such as in the presence of a given molecule factor (e.g., IPTG) or a given environmental condition (e.g., particular CO₂ concentration, nutrient levels, light, heat). In the absence of that condition, inducible promoters typically do not allow significant or measurable levels of transcriptional activity. For example, inducible promoters may be induced according to temperature, pH, a hormone, a metabolite (e.g., lactose, mannitol, an amino acid), light (e.g., wavelength specific), osmotic potential (e.g., salt induced), a heavy metal, or an antibiotic. Numerous standard inducible promoters will be known to one of skill in the art.

The recitation “polynucleotide” or “nucleic acid” as used herein designates mRNA, RNA, cRNA, rRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA and RNA.

The terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide, or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs that encode these enzymes.

With regard to polynucleotides, the term “exogenous” refers to a polynucleotide sequence that does not naturally-occur in a wild-type cell or organism, but is typically introduced into the cell by molecular biological techniques. Examples of exogenous polynucleotides include vectors, plasmids, and/or man-made nucleic acid constructs encoding a desired protein. With regard to polynucleotides, the term “endogenous” or “native” refers to naturally-occurring polynucleotide sequences that may be found in a given wild-type cell or organism. For example, certain plants do not typically contain a PAAS gene, and, therefore, do not comprise an “endogenous” polynucleotide sequence that encodes a PAAS polypeptide. Also, a particular polynucleotide sequence that is isolated from a first organism and transferred to second organism by molecular biological techniques is typically considered an “exogenous” polynucleotide with respect to the second organism. In specific embodiments, polynucleotide sequences can be “introduced” by molecular biological techniques into a plant cell that already contains such a polynucleotide sequence, for instance, to create one or more additional copies of an otherwise naturally-occurring polynucleotide sequence, and thereby facilitate overexpression of the encoded polypeptide.

“Polypeptide,” “polypeptide fragment,” “peptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In certain aspects, polypeptides may include enzymatic polypeptides, or “enzymes,” which typically catalyze (i.e., increase the rate of) various chemical reactions.

The recitation polypeptide “variant” refers to polypeptides that are distinguished from a reference polypeptide sequence by the addition, deletion or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted, or replaced with different amino acid residues.

The term “reference sequence” refers generally to a nucleic acid coding sequence, or amino acid sequence, to which another sequence is being compared. All polypeptide and polynucleotide sequences described herein are included as references sequences, including those described by name (e.g., a phenylacetaldehyde synthase, phenylacetaldehyde reductase, acyltransferase, propenylphenol synthase, allylphenol synthase, and etc.) and those described in the Sequence Listing.

The present disclosure contemplates the use in the methods described herein of variants of full-length enzymes or reference sequences, for instance, those having, phenylacetaldehyde synthase activity, phenylacetaldehyde reductase activity, acyltransferase activity, propenylphenol synthase activity, allylphenol synthase activity, among other reference sequences described herein, truncated fragments of these full-length enzymes and polypeptides, variants of truncated fragments, as well as their related biologically active fragments. Typically, biologically active fragments of a polypeptide may participate in an interaction, for example, an intra-molecular or an inter-molecular interaction. An inter-molecular interaction can be a specific binding interaction or an enzymatic interaction (e.g., the interaction can be transient and a covalent bond is formed or broken).

Biologically active fragments of a polypeptide/enzyme having a selected activity include peptides comprising amino acid sequences sufficiently similar to, or derived from, the amino acid sequences of a (putative) full-length reference polypeptide sequence. Typically, biologically active fragments comprise a domain or motif with at least one activity of a reference sequence or enzyme described herein, such as a phenylacetaldehyde synthase polypeptide, phenylacetaldehyde reductase polypeptide, acyltransferase polypeptide, propenylphenol synthase polypeptide, allylphenol synthase polypeptide, or a polypeptide associated with chemicals biosynthesis, and may include one or more (and in some cases all) of the various active domains. A biologically active fragment of such polypeptides can be a polypeptide fragment which is, for example, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200, 220, 240, 260, 280, 300, 320, 340, 360, 380, 400, 450, 500, 600 or more contiguous amino acids, including all integers in between, of a reference polypeptide sequence. In certain embodiments, a biologically active fragment comprises a conserved enzymatic sequence, domain, or motif, as described elsewhere herein and known in the art. Suitably, the biologically-active fragment has no less than about 1%, 10%, 25%, 50% of an activity of the wild-type polypeptide from which it is derived.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 97%, 98%, 99% or 100% sequence identity to any of the reference sequences described herein (see, e.g., Sequence Listing), typically where the polypeptide variant maintains at least one biological activity of the reference polypeptide.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Substantially” or “essentially” means nearly totally or completely, for instance, 95%, 96%, 97%, 98%, 99% or greater of some given quantity.

“Transformation” refers to the permanent, heritable alteration in a cell resulting from the uptake and incorporation of foreign DNA into the host-cell genome; also, the transfer of an exogenous gene from one organism into the genome of another organism.

By “vector” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated. Such a vector may comprise specific sequences that allow recombination into a particular, desired site of the host chromosome. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector can also include a selection marker such as an antibiotic resistance gene that can be used for selection of suitable transformants.

The term “wild-type” refers to a gene or gene product that has the characteristics of that gene or gene product when isolated from a naturally-occurring source. A wild-type gene or gene product (e.g., a polypeptide) is that which is most frequently observed in a population and is thus arbitrarily designed the “normal” or “wild-type” form of the gene.

As used herein, the term “woody plants,” refers to plants that produce wood as its structural tissue. Examples of woody plants include trees, shrubs, bushes, lianas, and etc.

As used herein, the term “fast growing tree/woody species,” refers to short-rotation woody plants. Examples of fast growing trees include Populus (e.g., poplars, aspen and etc.), Alnus (e.g., red alder, Caucasian alder, and etc.), Eucalyptus, willow, and etc.

As used herein, the term “Populus,” refers to a genus of 25-35 species of deciduous flowering plants in the family Salicaceae.

As used herein, the term “hybrid poplars,” refers to hybridization of two or more poplar species.

### Illustrated Embodiments

Embodiments of the present disclosure relate to modified plants, e.g., poplars, wherein the modified plant comprises one or more polynucleotides encoding one or more enzymes associated with chemical compound biosynthesis, such as aroma/fine/specialty compound biosynthesis. In some embodiments, the one or more polynucleotide(s) may be exogenous to the modified plant's native genome.

Embodiments of the present disclosure also relate to methods for generating modified plants, e.g., poplars, that produce chemical compounds, such as aroma/fine/specialty compounds, or produce an increased amount of the chemical compounds as compared with wide-type plants. Certain embodiments may include contacting one or more plant cells with one or more exogenous polynucleotides encoding one or more enzymes associated with chemical compound biosynthesis. The certain embodiments may also include cultivating the one or more plant cells for a time and under conditions sufficient to allow generation of the modified plants.

Embodiments of the present disclosure also relate to methods for producing chemical compounds, such as aroma/fine/specialty compounds, in modified plants, e.g., poplars. Certain embodiments may include introducing one or more exogenous polynucleotides encoding one or more enzymes associated with biosynthesis of the chemical compounds. The certain embodiments may also include cultivating a modified plant for a time and under conditions sufficient to allow production of the chemical compounds in the modified plants, and isolating the chemical compounds.

In some embodiments, the one or more enzymes may have a phenylacetaldehyde synthase (PAAS) activity and/or a phenylacetaldehyde reductase (PAR) activity. The present disclosure contemplates the use of naturally-occurring and/or non-naturally-occurring variants of these PAAS and/or PAR enzymes, as well as variants of encoding polynucleotides of these enzymes. In certain aspects, a PAAS encoding polynucleotide sequence is derived from Rosa hybrida cv. Fragrant Cloud phenylacetaldehyde synthase (RhFCPAAS) or Petunia hybrida cv. Mitchell phenylacetaldehyde synthase (PhMPAAS), and a PAR encoding polynucleotide sequence is derived from Lycopersicum esculentum phenylacetaldehyde reductase (LePAR1). In particular embodiments, the one or more enzymes may include both a PAAS activity and a PAR activity.

In some embodiments, the one or more enzymes may have an acyltransferase activity and/or a propenyl phenol synthase activity. The present disclosure also contemplates the use of naturally-occurring and/or non-naturally-occurring variants of these acyltransferase and propenyl phenol synthase enzymes, as well as variants of encoding polynucleotides of these enzymes. In certain aspects, an acyltransferase encoding polynucleotide sequence is derived from a Larrea tridentata acyltransferase (LtCAAT1), and a propenylphenol synthase encoding polynucleotide sequence is derived from a Larrea tridentata propenylphenol synthase (LtPPS1). In certain embodiments, the one or more enzymes may have an acyltransferase activity and a propenyl phenol synthase activity. In certain embodiments, the one or more enzymes may have at least one of a PAAS activity, a PAR activity, an acyltransferase activity, or a propenyl phenol synthase activity. In particular embodiments, the one or more enzymes may include a PAAS activity, a PAR activity, an acyltransferase activity, and a propenyl phenol synthase activity.

In some embodiments, the one or more enzymes may have an acyltransferase activity and/or an allyl phenol synthase activity. The present disclosure also contemplates the use of naturally-occurring and/or non-naturally-occurring variants of these acyltransferase and allyl phenol synthase enzymes, as well as variants of encoding polynucleotides of these enzymes. In certain aspects, an acyltransferase encoding polynucleotide sequence is derived from Larrea tridentata acyltransferase (LtCAAT1), and a propenylphenol synthase encoding polynucleotide sequence is derived from a Larrea tridentata allylphenol synthase (LtAPS1). In certain embodiments, the one or more enzymes may have an acyltransferase activity and an allyl phenol synthase activity. In certain embodiments, the one or more enzymes may have at least one of a PAAS activity, a PAR activity, an acyltransferase activity, or an allyl phenol synthase activity. In particular embodiments, the one or more enzymes may include a PAAS activity, a PAR activity, an acyltransferase activity, and an allyl phenol synthase activity.

In some embodiments, these enzyme encoding sequences may be derived from any organisms having a suitable PAAS, PAR, acyltransferase, propenyl phenol synthase, or allyl phenol synthase enzyme, and may also include any man-made variants thereof, such as any optimized coding sequences (i.e., codon-optimized polynucleotides) or optimized polypeptide sequences. Exemplary polypeptide and polynucleotide sequences are described infra.

In some embodiments, the modified plants may include two or more polynucleotides that encode an aroma/fine/specialty compound related enzyme (e.g., a PAAS, PAR, acyltransferase, propenyl phenol synthase, or allyl phenol synthase enzyme) or a variant or fragment thereof. For examples, the modified plants may include two or more polynucleotides that encode PAAS or a variant or fragment thereof. In particular embodiments, the two or more polynucleotides are identical or express the same PAAS. In certain embodiments, the two or more polynucleotides are different or may encode two different PAAS enzymes or a variant or fragment thereof. For example, in one embodiment, one of the polynucleotides may encode RhFCPAAS, while another polynucleotide may encode PhMPAAS.

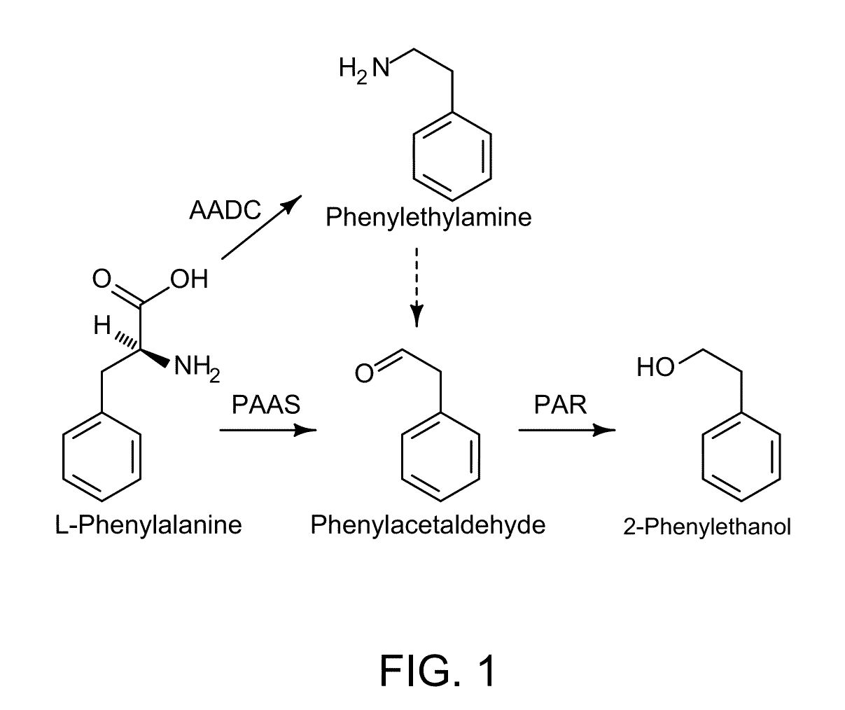

In some embodiments, the modified plants may include woody plants. In certain embodiments, the woody plants may include fast growing hardwood plant species, such as hybrid poplar, red alder, etc. These species offer the potential to serve as sustainable platforms for production of aroma, specialty and commodity chemicals, in addition to rapid biomass production. In particular embodiments, the fast growing hardwood plant species may be Populus tremula×P. alba or red alder, Alnus rubra. For example, hybrid poplar produces large amounts of biomass in a relatively short time period, and its fast growth, rapid propagation, and multiple harvests provide for an essentially unlimited potential of productivity. Its transformation efficiency and regeneration also lend it to expedient genetic engineering strategies. In particular embodiments, the carbon flux in hybrid poplars may be directed to produce 2-phenylethanol. As illustrated in FIG. 1, the biochemical pathway to produce 2-phenylethanol from phenylalanine may be introduced into hybrid poplar by transformation with genes encoding PAAS and phenylacetaldehyde reductase enzymes. The encoding genes may be overexpressed in hybrid poplars to produce 2-phenylethanol.

In some embodiments, the modified plants may function as a biotechnological platform for sustainably producing chemical compounds, such as commodity chemicals. The platform may enable development of further capability to produce these commodity chemicals and related intermediates and/or derivatives at scale and cost needed.

In some embodiments, the aroma/fine/specialty compounds may be sequestered by the modified plants as metabolites and carbohydrate or other covalent derivatives of the metabolites, thereby providing a mechanism for storage until needed. In certain embodiments, the aroma/fine/specialty compounds may be sequestered in non-volatile and/or non-toxic forms via derivatization in planta (e.g., glycosylation, acylation, and etc.) such that these compounds are stored or transported until needed. For example, 2-phenylethanol produced by the modified plants under control of the CaMV 35S promoter may be sequestered via glycosylation or acylation, a mechanism for plant storage of several secondary metabolites and also for detoxification in planta.

In some embodiments, the aroma/fine/specialty compounds may be 2-phenylethanol. 2-phenylethanol is a colorless liquid and may be found in the essential oils of several plant species. 2-phenylethanol has a “rose-like” aroma and is widely used as a fragrance and flavoring agent, as an antibiotic agent, and also for the production of derivatives such as the flavoring agent phenyl ethyl-acetate. Much of 2-phenylethanol's commercial production is performed through chemical synthesis from petrochemical products, benzene or styrene. In some instances, 2-phenylethanol may be converted into styrene and ethyl benzene in vitro.

Several attempts to biotechnologically produce 2-phenylethanol in microorganisms have been made, but with no technical/commercial success due to, for example, poor yields. For example, utilizing mainly the Ehrlich pathway of amino acid catabolism, maximal productivity levels obtained so far in S. cerevisiae reach around 5 g/l and up to 25 g/L with Kluyveromyces marxianus. Complex culture techniques increase costs, which make the endeavor economically unfavorable. An adverse factor in biotechnological 2-phenylethanol production may be related to 2-phenylethanol's inherent antimicrobial activity as it is neither sequestered as a stable (non-toxic) form nor stored in a protective compartment (e.g., cell wall, vacuole, etc.).

In certain plants, 2-phenylethanol may be produced via two complementary biosynthetic pathways. The first one includes an initial decarboxylation step, catalyzed by an aromatic amino acid decarboxylase (AADC), and an oxidative deamination step. A second two step pathway includes an initial step catalyzed by the bifunctional PAAS that engenders sequential decarboxylation and oxidation of phenylalanine forming phenylacetaldehyde, with the latter then reduced by PAR to afford 2-phenylethanol. A PAAS resulted from searching expressed sequence tag databases from both Petunia hybrida and Rosa hybrida for sequences is similar to aminotransferases, decarboxylases and amine/monoamine oxidases. Of the possible candidates may be two homologs (one from petunia and one from rose) to L-tyrosine/3,4-dihydroxy-L-phenylalanine (L-Dopa) decarboxylase (DDC) having expression patterns correlated temporally and spatially with 2-phenylethanol production. PAAS is a bifunctional enzyme catalyzing both decarboxylation and O₂-coupled oxidation generating phenylacetaldehyde, CO₂, ammonia and hydrogen peroxide.

In some embodiments, a plant may be genetically modified according to routine techniques known in the art, such as by Agrobacterium mediated transformation of vectors suitable for use in the modified plant. In certain aspects, genetic manipulation in the plant can be performed by introduction of non-replicating transfer DNA (T-DNA) region in the vector that contain native plant's sequences, exogenous genes of interest, and drug resistance genes. Upon introduction into the plant cell, the T-DNA may be integrated into the plant's genome through homologous recombination. In this way, the exogenous gene of interest and the drug resistance gene are stably integrated into the plant's genome. Examples of suitable vectors are provided herein.

For an example, a Populus tremula×alba may be genetically modified by introduction of a pKGW vector harboring both PAAS (RhFCPAAS from Rosa hybrida cv. Fragrant Cloud or PhMPAAS from Petunia hybrida cv. Mitchell) and PAR (LePAR, from Solanum lycopersicum). This may be carried out under the control of a promoter (e.g., the CaMV 35S promoter), and thus the biosynthetic pathway may be directed from (endogenous) phenylalanine to afford 2-phenylethanol after functional expression of these exogenous genes.

In certain embodiments, the modified plant may be prepared by (i) introducing one or more desired polynucleotides encoding the one or more enzymes associated with chemical compound biosynthesis, such as aroma compound biosynthesis, (ii) selecting for, and/or isolating, plants that comprise the one or more desired polynucleotides. For an example, selection and isolation may include the use of antibiotic resistant markers known in the art (e.g., kanamycin, spectinomycin, and streptomycin). For example, a poplar may be co-cultivated with Agrobacterium containing the RhFCPAAS/LePAR1 construct. The poplar may produce numerous shoots from calli within a predetermined time (e.g., 8 weeks). Using genomic PCR screening, the regenerated shoots selected on kanamycin containing the transgenes of interest may be determined.

In a particular embodiment, a poplar transformed with both the pKGW::RhFCPAAS/LePAR and pKGW::PhMPAAS/LePAR constructs may accumulate high transcript levels of both PAAS and PAR, and also produce and accumulate 2-phenylethanol and its glycosylated derivative 2-phenylethyl-glucoside.

In some embodiments, the one or more enzymes may have a PAAS activity that is optimized to produce an increased amount of 2-phenylethanol as compared with non-optimized PAAS enzymes. For example, amino acid sequences of the one or more polynucleotides may be modified to contain or may contain the phenylalanine residue associated with oxidative decarboxylation activity. In certain embodiments, the amino acid sequence for PAAS activity may be optimized based on the rose homolog to obtain a higher PAAS activity. In certain embodiments, the PAAS oxidative decarboxylation step may be the rate-limiting factor in the synthesis of 2-phenylethanol.

In some embodiments, the chemical compounds and related metabolites may be extracted and analyzed. In certain embodiments, the chemical compounds and related metabolites may be extracted by (i) grinding materials (e.g., a stem, leave, and etc.) of the modified plant that have been frozen, (ii) extracting aroma/fine/specialty compounds such as 2-phenylethanol, eugenol, etc., using ether, or an aqueous miscible solvent (if covalently linked to a sugar) followed by chemical or enzymatic hydrolysis or hydrolysis, or by steam distillation or distillation or extraction.

In some embodiments, a spatial location of 2-phenylethanol and/or 2-phenylethyl glycoside in situ on older leaves and/or cryo-sectioned stems of transgenic hybrid poplar lines may be identified using MALDI imaging mass spectrometry. In certain embodiments, 2-phenylethanol and/or 2-phenylethyl glycoside may be abundantly distributed throughout the leaf including the lateral veins and midrib. In particular embodiments, 2-phenylethanol and/or 2-phenylethyl glycoside may also be distributed in stems.

In some embodiments, a modified microorganism (e.g., E. coli) may comprise one or more polynucleotides encoding one or more enzymes associated with a chemical compound. The microorganism may be genetically modified to produce the aroma compounds. For example, either the Rosa hybrida cv. Fragrant Cloud phenylacetaldehyde synthase (RhFCPAAS) or the Petunia hybrida cv. Mitchell phenylacetaldehyde synthase (PhMPAAS) simultaneously with a tomato (Lycopersicum esculentum) phenylacetaldehyde reductase (LePAR1) may be introduced in an E. coli system to produce 2-phenylethanol and/or related derivatives.

The various embodiments described above can be combined to provide further embodiments. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### EXAMPLES

### Example 1

### RNA Extraction and cDNA Preparation

Total RNA was individually isolated from 100 mg of flash-frozen petal limb tissue of a partially open stage 4 Rosa hybrida “Fragrant Cloud” rose bud, early-stage flowering Petunia hybrida cv. Mitchell corolla limbs, and Lycopersicum esculentum tomato young leaf, respectively, using a SPECTRUM™ PLANT TOTAL RNA KIT (Sigma) according to the manufacturer's instructions. After DNAse I treatment (Deoxyribonuclease I, Amplification Grade (Invitrogen™)), first strand cDNA synthesis was performed using an aliquot (1 μg) of the total RNA with a SuperScript™ III First-Strand Synthesis System for RT-PCR (Invitrogen) according to the manufacturer's instructions.

### Example 2

### Gene Cloning

The RhFCPAAS and PhMPAAS genes were amplified from rose petal limb, or petunia corolla limb, cDNA using the primers RhFCPAAS-F and RhFCPAAS-R, or PhMPAAS-F and PhMPAAS-R, respectively (Table 1), designed from the published cDNA sequences for Rosa hybrida phenylacetaldehyde synthase (RhPAAS) (GenBank accession number DQ192639) and Petunia hybrida cv. Mitchell (GenBank accession number DQ243784). The LePAR1 cDNA was amplified using primers designed based on the published cDNA sequence (GenBank accession number EF613490). Amplification was performed using Pfu Turbo® DNA Polymerase (Stratagene) in a PCR mix containing 1 μl of the cDNA preparation. PCR amplification was accomplished using a Bio-Rad C1000 Thermal Cycler with an initial denaturation step of 95° C. for 2 min followed by 35 cycles of 94° C. denaturing for 30 s, 55° C. annealing for 30 s, and 70° C. extension for 3 min, with a final extension at 70° C. for 10 min. The PCR products were resolved using UltraPure™ L.M.P. Agarose gel (Invitrogen™), where a single band of approximately 1500 bp for the PAAS, or 1000 bp for the PAR1, was obtained. The RhFCPAAS, PhMPAAS, and LePAR1 gene products purified from the PCR gel were cloned into pENTR™/D-TOPO® (Invitrogen™) for sequence confirmation.

TABLE 1

| Primer name | Sequence | SEQ ID |
|---|---|---|
| RhFCPAAS-F | (CACC)ATGGGT | SEQ ID |
| | AGCTTCCCATTC | NO: 1 |
| | CAC | |
| RhFCPAAS-R | TCAATACGTGCT | SEQ ID |
| | GAGGATTGC | NO: 2 |
| RhFCPAAS-XHOI-F | (CACC)CTCGAG | SEQ ID |
| | ATGGGTAGCTTC | NO: 3 |
| | CCATTCC | |
| RhFCPAAS-BGLII-R | AGATCTTCAATA | SEQ ID |
| | CGTGCTGAGGAT | NO: 4 |
| | TGCTTG | |
| (RhFCPAAS)- | GGGGACAACTTT | SEQ ID |
| attB1-p35S-F | GTATACAAAAGT | NO: 5 |
| | TGTCGACGAATT | |
| | AATTCCAATCCC | |
| | ACA | |
| (RhFCPAAS)- | GGGGACCACTTT | SEQ ID |
| attB5r-tOCS-R | GTACAAGAAAGC | NO: 6 |
| | TGGGTAAGATTT | |
| | AGGTGACACTAT | |
| | AGAATATGCATC | |
| | ACTAGTAAGCTA | |
| | GC | |
| LePAR1-F | (CACC)ATGAGT | SEQ ID |
| | GTGACAGCGAAA | NO: 7 |
| | ACA | |
| LePAR1-R | TTACATAGAAGA | SEQ ID |
| | TGAACCTCC | NO: 8 |
| (LePAR1)-attB5- | GGGGACAAGTTT | SEQ ID |
| p35S-F | GTACAAAAAAGC | NO: 9 |
| | AGGCTCTAGAGC | |
| | CAAGCTGATCTC | |
| | CT | |
| (LePAR1)-attB2- | GGGGACAACTTT | SEQ ID |
| t35S-R | TGTATACAAAGT | NO: 10 |
| | TGTCTAGAGGGC | |
| | CCGACGTCGCAT | |
| LePAR1-QPCR-F | TGTGAAGGCTTC | SEQ ID |
| | TGTTCGTG | NO: 11 |
| LePAR1-QPCR-R | GCACATGACCCG | SEQ ID |
| | AGAAGATT | NO: 12 |
| PtaACTIN-QPCR-F | GGTCCTCTTCCA | SEQ ID |
| | ACCTTCAA | NO: 13 |
| PtaACTIN-QPCR-R | TCCTGGGAACAT | SEQ ID |
| | AGTTGAACC | NO: 14 |

### Example 3

### Expression in E. coli

RhFCPAAS and LePAR1 genes were cloned in tandem into a pETDUET-1 expression vector (Novagen) to study the metabolites produced resulting from co-expression of the two target genes. To prepare the expression vector, the LePAR1 cDNA was first PCR amplified to include Nde I and Xho I restriction enzyme sites at the 5′ and 3′ ends, respectively, cloned into a pCR4®-TOPO® vector (Invitrogen™) and then cut out and cloned into these sites in the MCS2 region of the pETDUET-1 vector. The RhFCPAAS cDNA was PCR amplified with primers that included BspH I and Not I restriction enzyme sites and was cloned into a pENTR™/D-TOPO® vector (Invitrogen™). The adapted RhFCPAAS was then cut out from the pENTR™/D-TOPO® vector construct using the BspH I and Not I restriction enzyme sites and ligated into the compatible Nco I and Not I sites in the MCS1 region of the previous LePAR1/pETDUET construct. A similar cloning scheme was used to incorporate the PhMPAAS cDNA into the LePAR1/pETDUET-1 vector, except that the PCR adapter restriction enzyme cloning sites to clone into the MCS1 were BspH I and Sac I. Competent BL21 (DE3) Singles™ E. coli cells (Novagen®) were transformed with 20 ng of the final plasmid preparations. With the empty pETDUET-1 vector used as a control, 50 ml LB_{carbenicillin100} cultures of each expression vector assembly were incubated at 37° C. to an OD₆₀₀ of 0.8. The cultures were induced with IPTG to a final concentration of 1.0 mM, in the presence of phenylalanine (5 μm), pelleted, with the supernatants extracted with hexane (1 ml) containing 0.5 mM benzyl-methyl-ether as internal standard and analyzed directly via GC-MS.

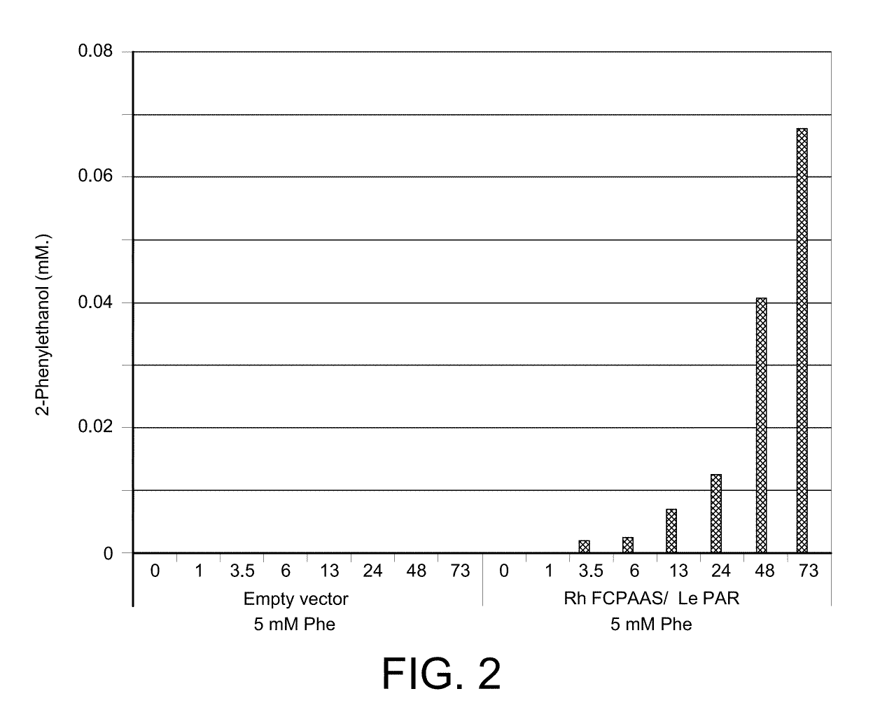

The rose RhFCPAAS gene were expressed simultaneously with tomato LePAR in an E. coli system to provide synchronized expression of the PAAS and PAR genes that result in the production of 2-phenylethanol. When E. coli expressing RhFCPAAS/LePAR were grown in the presence of phenylalanine (5 mM) after induction by IPTG, it indicated the successful production of 2-phenylethanol in the culture medium, reaching a concentration of 0.07 mM after 73 hour cultivation. As illustrated in FIG. 2, phenylacetaldehyde accumulation at 73 hours was of only 0.9 μM.

### Example 4

### Vector Construction for Over-Expression in Hybrid Poplar

The 1.5 kb RhFCPAAS and PhMPAAS cDNA were next amplified with primers that included 5′-Xho I or 3′-Bgl II restriction enzyme sites for subsequent cloning into the pART7 vector (Table 1, primers RhFCPAAS-XHOI-FOR and RhFCPAAS-BGLII-REV, or PhMPAAS-XHOI-FOR and PhMPAAS-BGLII-R, respectively). The PCR products were separated on and eluted from an UltraPure™ L.M.P. agarose gel and were again cloned into pENTR™/D-TOPO® vector (Invitrogen™). Following sequencing confirmation, the RhFCPAAS and PhMPAAS cDNA were cut out using a double digest of Xho I and Bgl II restriction endonucleases and, after gel purification, ligated into the pART7 vector in the MCS region Xho I and BamH I sites.

A new set of primers was designed to amplify the fragment of the pART construct to contain the CaMV 35S promoter, the RhFCPAAS cDNA and the OCS terminator, with adapters on each end for use in a BP clonase recombination with pDONR221 P1-P5r vector (See Table 1, primers (RhFCPAAS)-attB1-p35S-F and (RhFCPAAS)-attB5r-tOCS-R). The BP clonase reaction was carried out according to the Invitrogen™ protocol for a Gateway pDONR recombination reaction and included 3 μl attB PCR product, 1 μl pDONR221 plasmid, and 2 μl BP Clonase II enzyme mix. This same procedure was used to clone the PhMPAAS cDNA into the pDONR221 P1-P5 vector.

The LePAR1/pENTR™/D-TOPO® construct was recombined into the Gateway binary vector pK2GW7 using Gateway LR Clonase II (Invitrogen™). Primers were then designed to amplify the p35S::LePAR1:t35S fragment from the LePAR1-pK2GW7 construct (See Table 1, primers (LePAR1)-attB5-p35S-F and (LePAR1)-attB2-t35S-R) and included adapter sequences for BP Clonase recombination into the pDONR221 P5-P2 vector (Invitrogen™).

The RhFCPAAS-pDONR221 P1-P5r, or the PhMPAAS-pDONR221 P1-P5r, and LePAR1-pDONR221 P5-P2 constructs were subsequently cloned in a two-fragment MultiSite Gateway® Pro Plus Kit (Invitrogen) recombination scheme into the promoter-less Gateway binary vector pKGW with a Gateway 2-fragment recombination reaction using LR Clonase II Plus enzyme (Invitrogen™) according to the manufacturer's instructions. The final recombined construct was transformed into E. coli and plated onto LB_{Spectinomycin50} medium.

All plasmids were prepared using the Wizard Plasmid DNA Kit (Promega), and these were propagated using One Shot Mach1™-T1® chemically competent E. coli (Invitrogen™). The final Gateway constructs propagated in E. coli were subjected to DNA sequencing to confirm correct insertion of all gene fragments-of-interest in the T-DNA binary vector, with these then introduced into Agrobacterium tumefaciens (A. tumefaciens) strain LBA4404 using a freeze thaw method. Transformed A. tumefaciens colonies were screened by PCR using appropriate transgene and vector specific primer combinations to confirm that the correct T-DNA vectors had been assimilated.

### Example 5

### Poplar Transformation

Hybrid poplar Populus tremula×P. alba stem and leaf pieces were transformed by co-cultivation with A. tumefaciens containing either the RhFCPAAS/LePAR1 or the PhMPAAS/LePAR1 constructs or an empty vector control, using standard methods. Shoots were generated and selected on solid agar medium (Agar-TC, Phytotechnologies, Inc.) containing kanamycin sulfate (GIBCO/Invitrogen) at a final concentration of 100 μg/ml. Transformed shoots were identified initially by survival and growth on medium containing a kanamycin selectable marker. Genomic DNA from regenerated plants was assayed directly by PCR screening using a REDExtract-N-Amp Plant PCR Kit (Sigma) to confirm the stable integration of the T-DNA containing the transgenes into the poplar genome. After a three month selection process, plants that contained the introduced genes-of-interest were then transferred to the greenhouse.

The transgenic poplar plants appeared to grow normally with no distinct phenotypic alterations compared to wild type or to control plants transformed with Agrobacterium containing the empty binary vector. Starting from an early shoot development stage while the shoots were still in agar medium in enclosed sterile containers, a distinct rose floral scent was noticeable emanating from the transgenic plants. Furthermore, trees transformed with both the pKGW::RhFCPAAS/LePAR and pKGW::PhMPAAS/LePAR constructs were shown to accumulate high transcript levels of both PAAS and PAR, and also to produce and accumulate 2-phenylethanol and its glycosylated derivative 2-phenylethyl-glucoside.

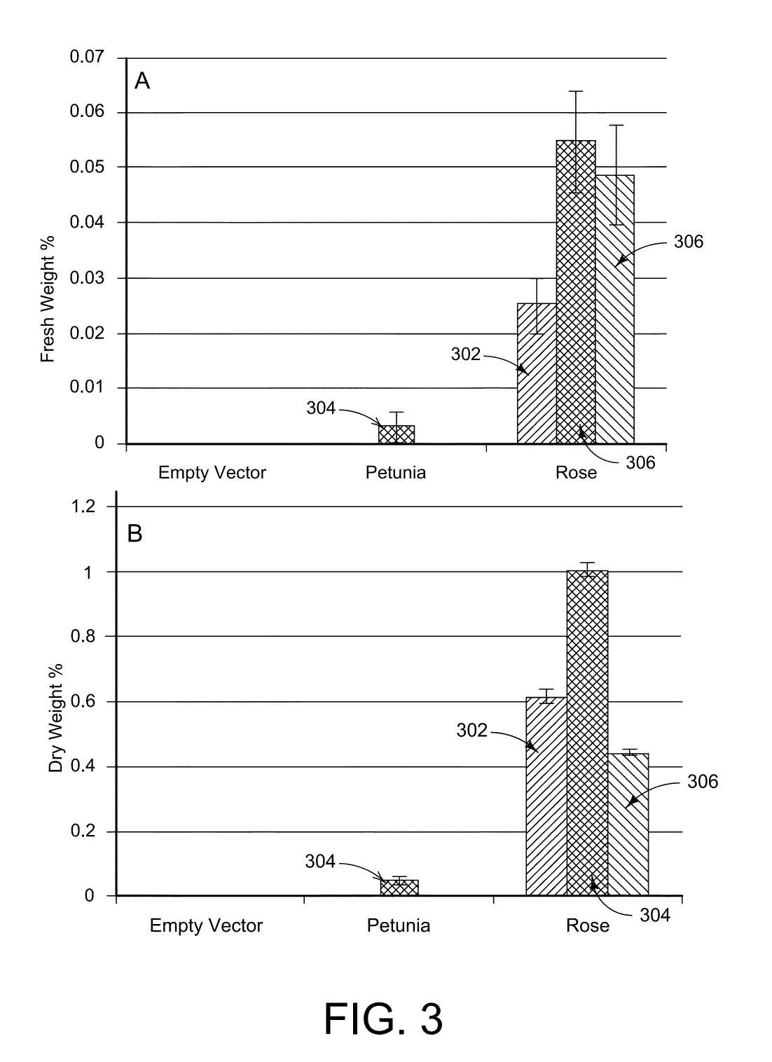

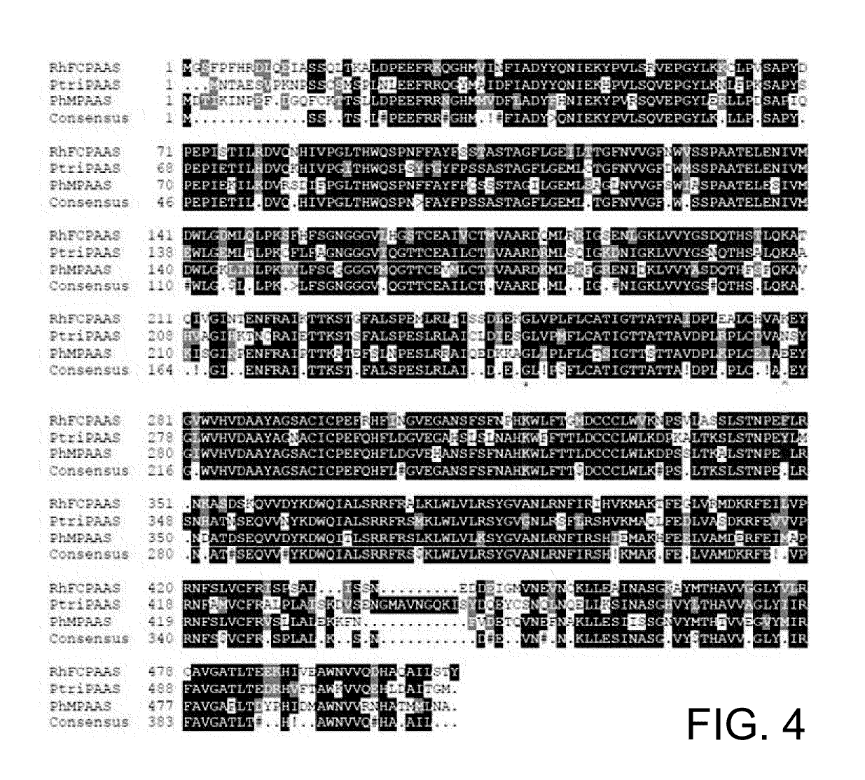

Poplar trees transformed with both constructs had relatively similar expression levels, the plants expressing PAAS from Petunia hybrida cv. Mitchell accumulated significantly lower amounts of 2-phenylethanol and 2-phenylethyl-glucoside than the plants expressing the gene obtained from Rosa hybrida cv. Fragrant Cloud (FIG. 3). Alignment of the synthase homologues from various species, including rose, petunia, and poplar (FIG. 4) indicated that, although all homologues contain the necessary pyridoxal 5′-phosphate (PLP)-binding lysine residue, only the rose amino acid sequence contains the phenylalanine residue associated with oxidative decarboxylation activity.

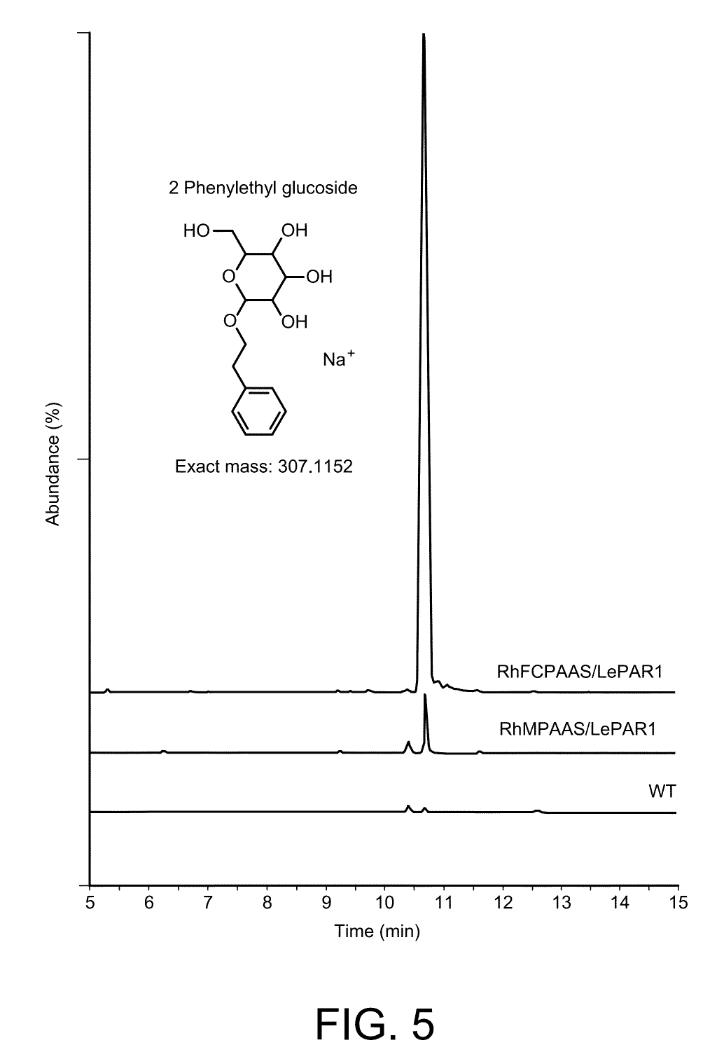

Accumulation was, however, time dependent with significantly higher metabolite levels in older leaves. These results indicate that 2-phenylethyl-glucoside, as a more hydrophilic and stable compound, acts as a sink for 2-phenylethanol production leading to higher carbon flow through the pathway and with no detrimental effects to physiology and morphology observed. The lines investigated so far have thus shown accumulation levels reaching 0.05% in fresh weight for 2-phenylethanol in older leaves (FIG. 3A) and with its derivative 2-phenylethyl-glucoside (FIG. 5) in levels reaching 1% dry weight (FIG. 3B).

### Example 6

### Real-Time QPCR Analysis

Total RNA from poplar plants was prepared using the SPECTRUM™ PLANT TOTAL RNA KIT (SIGMA). An aliquot (2 μm) of the total RNA was subsequently used for cDNA preparation. After DNAse I treatment (Deoxyribonuclease I, amplification grade (Invitrogen)), an aliquot (2 μm) of the total RNA was used to perform RT-PCR using the SuperScript™ III First-Strand Synthesis System for RT-PCR (Invitrogen™). Gene amplification was performed using QPCR Platinum SYBR® Green qPCR SuperMix-UDG (Invitrogen™) for the qPCR reactions. The reaction mix included forward and reverse gene-specific primers, buffer, and ROX Reference Dye. A Stratagene Mx3005p QPCR System was used for qPCR gene amplification. The internal reference gene control was an actin gene isolated from the hybrid poplar. Primer sequences used for real-time qPCR are shown in Table 1 as RhFCPAAS-QPCR-F and RhFCPAAS-QPCR-R for the rose synthase, PhMPAAS-QPCR-F and PhMPAAS-QPCR-R for the petunia synthase, LePAR1-QPCR-F and LePAR1-QPCR-R for the tomato reductase, and PtaACTIN-QPCR-F and PtaACTIN-QPCR-R for the reference actin gene. After an initial screening of transgenic plants compared to a wild type plant, a transgenic plant expressing the lowest levels of RhFCPAAS, or PhMPAAS, and LePAR1 genes compared to the wild type plant was selected as the control calibrator in all future qPCR comparisons of the respective transgenes. Reactions were performed in triplicate using 2 μl of a 1:5 dilution of the First-Strand cDNA preparation.

### Example 7

### Metabolite Extraction and Analysis

Plant material was flash frozen in liquid nitrogen, ground using a 5-mm steel ball in a Tissue-lyzer (Qiagen) still frozen and kept at −80° C. until extraction. For free 2-phenylethanol profiling, samples were extracted with 2 μl/mg of methyl-tert-butyl ether containing 0.5 mM benzyl-methyl-ether as internal standard and extracts analyzed in a HP 6890 Series GC System equipped with a RESTEK-5Sil-MS (30 m×250 μm×0.25 μm) column. The temperature program used was as follows: 40° C. maintained for 2 min and then raised from 0 to 150° C. at 10° C./min, then from 150 to 250° C. at 20° C./min with a final holding time of 2 min; total run time 22 min. Injector and detector temperatures were set at 250 and 230° C., respectively. 2-Phenylethanol was quantified based on m/z 122 and 91 extracted ion traces.

For 2-phenylethyl glucoside determinations, samples were extracted with methanol:water (7:3, v/v) containing 0.5 mM naringenin as internal standard. Extracts were analyzed using a Waters Acquity ultra performance liquid chromatography system, equipped with a Waters BEH C18 column (1.7 μm particles, 2.1×100 mm) with a binary mobile phase of water with 0.1% formic acid (A) and acetonitrile with 0.1% formic acid (B), with detection electrospray ionization mass spectrometry in the positive mode in a Waters Xevo G2 Q-TOF mass spectrometer using as a lock-mass standard leucine-enkephalin, at a capillary voltage of 3 kV, cone voltage of 38 V, a desolvation gas temperature of 280° C. and source temperature of 100° C. The gradient program was as follows: flow rate of 0.2 ml/min; linear gradient of water with 0.1% formic acid:acetonitrile with 0.1% formic acid from 95:5 to 65:35 in 35.5 min, to 0:100 in 1 min, followed by 2 min at 0:100 and re-equilibration at initial conditions for 3 min. The column temperature was held at 25° C. and sample injection volume was 1 μl. Detection and quantification of 2-phenylethylethanol-glucoside was performed by integrating the corresponding peak with retention time of 10.7 minutes in the extracted m/z 307.116 (corresponding to [M+Na]⁺) chromatogram.

### Example 8

### Allyl/Propenylphenol Synthesis in Transgenic Poplars

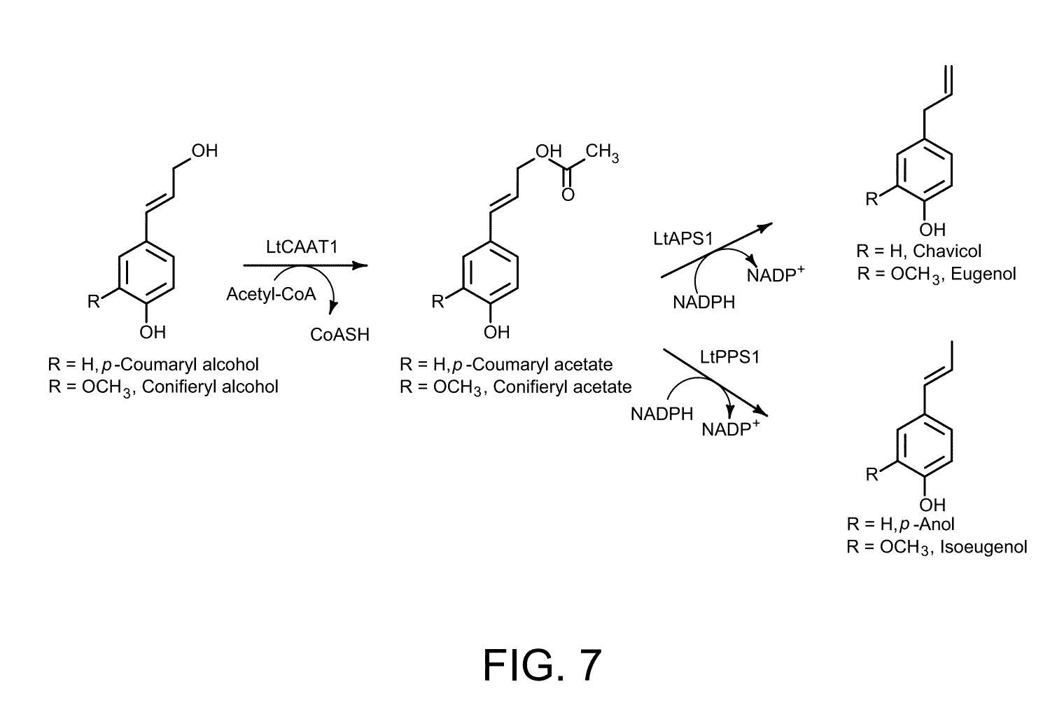

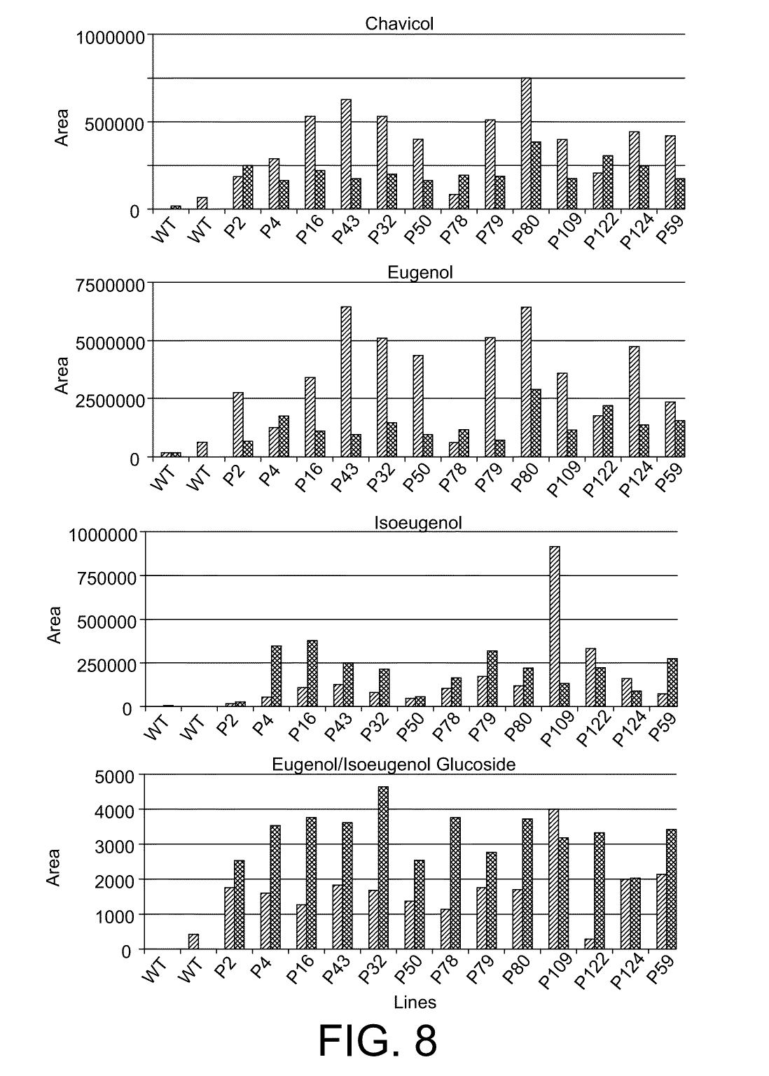

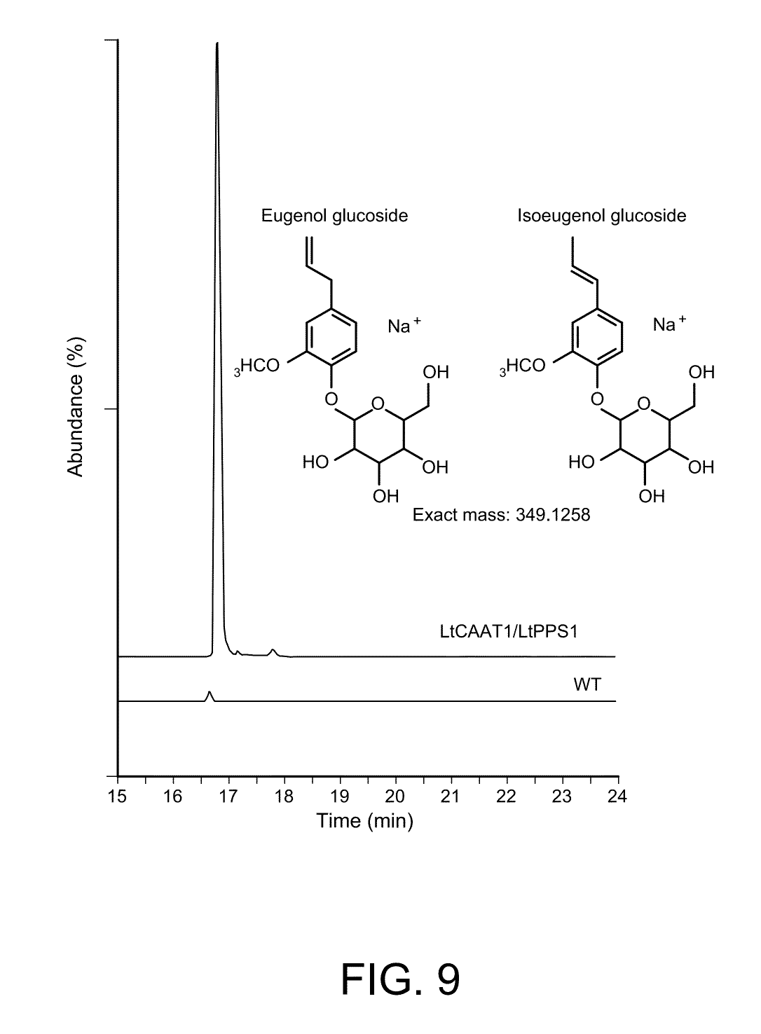

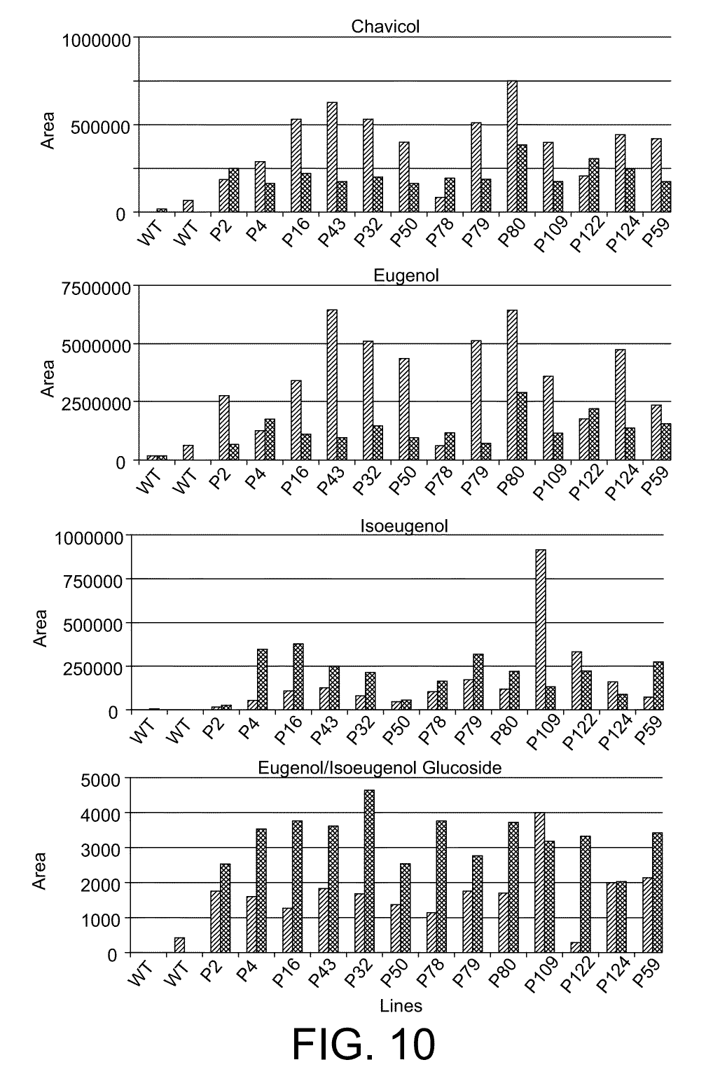

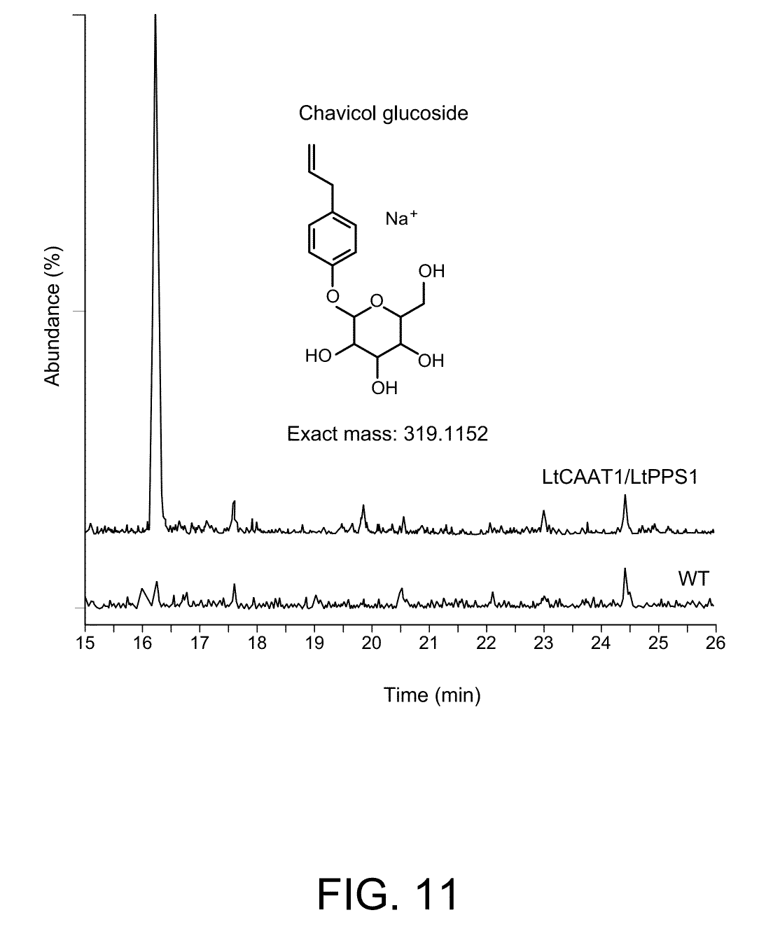

Hybrid poplar was also transformed with constructs containing an acyltransferase (LtCAAT1) and a propenylphenol synthase (LtPPS1) or LtCAAT1 and an allylphenol synthase (LtAPS1), with all genes cloned from the creosote bush (Larrea tridendata). This was carried out in order to convert monolignols to allyl/propenyl phenols (e.g. chavicol/eugenol and p-anol/isoeugenol, FIG. 7) which can then be used after modifications as biofuel constituents and other end-use bioproducts. Thus, the constructs, pK2GW7/35S::LtCAAT1::35S::LtAPS1, and pK2GW7/35S::LtCAAT1::35S::LtPPS1 were generated and individually transformed into hybrid poplar (Populus tremula×P. alba), this being mediated by Agrobacterium tumefaciens EHA105 as described above for 2-phenylethanol (glucoside) production. pK2GW7/35S::LtCAAT1::35S::LtAPS1 and pK2GW7/35S::LtCAAT1::35S::LtPPS1 lines were generated including trees. Trees transformed with either pK2GW7/35S::LtCAAT1::35S::LtAPS1 or pK2GW7/35S::LtCAAT1::35S::LtPPS1 constructs were shown to accumulate high transcript levels of both LtCAAT1 and LtAPS1/LtPPS1, as well as producing and accumulating allyl/propenylphenols and their glycosylated derivatives as shown FIGS. 8-11.

### Example 9

### MALDI Imaging

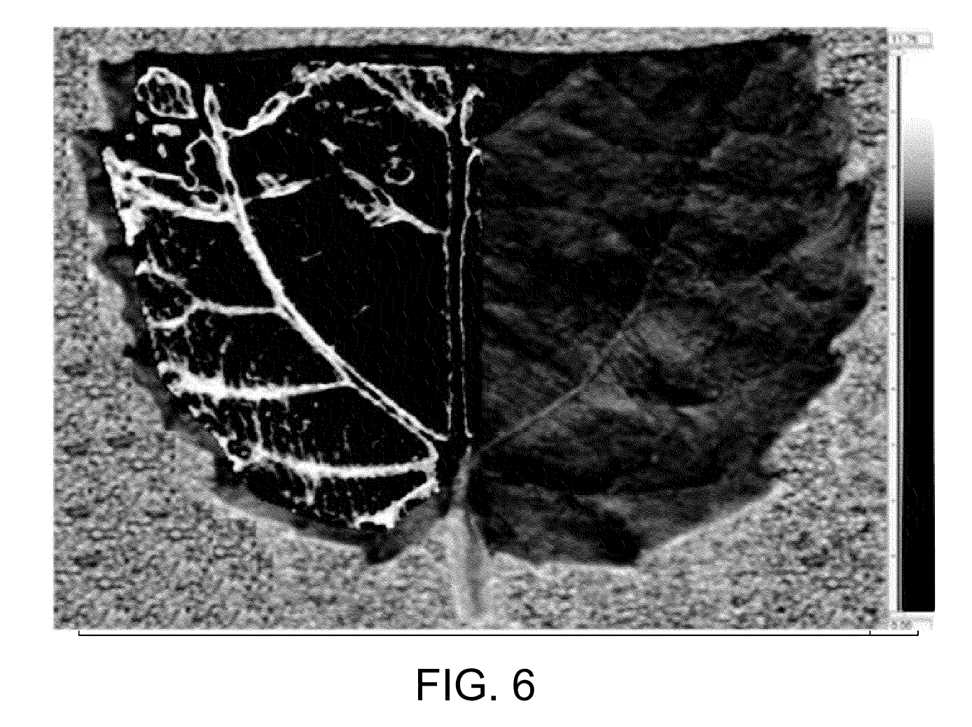

The spatial localization of 2-phenylethyl glycoside in situ on both older leaves and cryo-sectioned stems of transgenic hybrid poplar lines were mapped and established using MALDI imaging mass spectrometry. With synthetic 2-phenylethyl glucoside as a standard, ions m/z 307.11 and 323.09 corresponding to [M+Na]⁺ and [M+K]⁺ were readily detected and identified with 2,5-dihydroxybenzoic acid (DHB) as matrix. Next, specific localization of 2-phenylethyl glucoside in the transgenic poplar lines (leaf and stem tissues) was detected via its K adduct ion (m/z 323.09). The high potassium adduct ion intensity observed in transgenic poplar leaf and stem tissues may provisionally be explained by the high concentration of potassium salts in the analyzed tissues thus making it more readily available for adduct formation during analyte ionization. Specifically, a section leaf from poplar hybrid gene expressing PAAS from Rosa hybrida cv. Fragrant Cloud) gave hundreds of metabolites ranging from m/z 100 to 700 Da, with the most intense and abundant peak being the K adduct ion (m/z 323.09). As illustrated in FIG. 6, it is abundantly distributed throughout the leaf but mainly in the lateral veins and midrib. By contrast, while the poplar hybrid leaf expressing the petunia PAAS also showed hundreds of metabolites with mass ranges between m/z 100 to 700 Da, the 2-phenylethyl glucoside K adduct ion was less intense (ca. 11%). The leaf from a hybrid poplar transformed with an empty vector; also showed hundreds of metabolites but none corresponded to 2-phenylethyl glucoside. These results are thus in concurrence with the data acquired from the UPLC-MS analysis of the leaf crude extracts from the poplar hybrids (FIG. 3).

Further, the localization of 2-phenylethyl glucoside in the stems of transgenic poplar hybrids using 20 μm cryosectioned stem tissues (cross- and longitudinal sections) was mapped. Similar to the transgenic poplar leaves, the stems also contained hundreds of metabolites, including the 2-phenylethyl glucoside ion at m/z 323.09, [M+K]⁺. However, the ion intensity was weak and only 3-10% abundant, in contrast to the leaves. In addition, it is distributed moderately in tissues of stem expressing the rose PAAS, where it is apparently localized specifically in epidermal tissues. In contrast to the stem of poplar hybrid expressing the petunia PAAS, 2-phenylethyl glucoside is sparingly distributed in the stem tissues, and is absent in poplar stems transformed with an empty vector.

1-82. (canceled)

83. A modified plant comprising one or more exogenous polynucleotides encoding one or more enzymes associated with an aroma compound biosynthesis, a fine compound biosynthesis, a specialty compound biosynthesis or a combination thereof.

84. The modified plant of claim 83, wherein the one or more enzymes is a synthase, a reductase, a transferase, or a combination thereof.

85. The modified plant of claim 84, wherein
the synthase is a phenylacetaldehyde synthase, a propenylphenol synthase, an allylphenol synthase, a biologically active fragment or variant thereof or a combination thereof;
the reductase is a phenylacetaldehyde reductase or a biologically active fragment or variant thereof; and
the transferase is an acyl transferase or a biologically active fragment or variant thereof

86. The modified plant of claim 85, wherein the propenyl phenol synthase is Larrea tridentate propenylphenol synthase or a biologically active fragment or variant thereof and the allylphenol synthase is Larrea tridentata allylphenol synthase or a biologically active fragment or variant thereof.

87. The modified plant of claim 83, wherein the aroma compounds include at least one or more metabolites associated with 2-phenyl ethanol synthesis.

88. The modified plant of claim 83, wherein the modified plant produces a 2-phenyl ethanol, an allyl phenol, a propenyl phenol or a combination thereof.

89. The modified plant of claim 83, wherein the modified plant is selected from the group consisting of a poplar, a poplar hybrid, an aspen, and a red alder.

90. The modified plant of claim 83, wherein the modified plant produces aroma compounds that are sequestered in the modified plant as aroma compounds, covalent derivatives of one or more aroma compounds or both.

91. The modified plant of claim 90, where the one or more aroma compounds includes:
2-phenylethanol that is sequestered as a 2-phenylethyl-glucoside or other ethylated form;
an allyl phenol; or
propenyl phenol.

92. A method for producing a modified plant, the method comprising contacting one or more plant cells with one or more exogenous polynucleotides encoding one or more enzymes associated with aroma compounds biosynthesis; and cultivating the one or more plant cells to generate the modified plant.

93. The method of claim 92, wherein the one or more enzymes is a synthase, a reductase, a transferase or a combination thereof.

94. The method of claim 93, wherein
the synthase is a phenylacetaldehyde synthase, a propenylphenol synthase, an allylphenol synthase, a biologically active fragment or variant thereof or a combination thereof;
the reductase is a phenylacetaldehyde reductase (PAR) wherein the PAR is lycopersicum esculetum phenylacetaldehyde reductase (LePAR1) or a biologically active fragment or variant thereof; and.
the transferase is an acyl transferase (ltCAAT1) or a biologically active fragment or variant thereof.

95. The method of claim 94, wherein the propenyl phenol synthase is Larrea tridentate propenylphenol synthase or a biologically active fragment or variant thereof and the allylphenol synthase is Larrea tridentata allylphenol synthase or a biologically active fragment or variant thereof.

96. The method of claim 92, wherein the aroma compounds include at least one or more metabolites associated with 2-phenyl ethanol synthesis.

97. The method of claim 92, wherein the modified plant produces a 2-phenyl ethanol, an allyl phenol, a propenyl phenol or a combination thereof.

98. The method of claim 92, wherein the wherein the modified plant produces aroma compounds that are sequestered in the modified plant as aroma compounds, covalent derivatives of one or more aroma compounds or both.

99. The method of claim 98, where the one or more aroma compounds include:
2-phenylethanol that is sequestered as a 2-phenylethyl-glucoside or other ethylated form;
an allyl phenol; or
propenyl phenol.

100. A method for producing aroma compounds from a modified plant, the method comprising: cultivating a modified plant to produce the aroma compounds, wherein the modified plant includes one or more exogenous polynucleotides encoding one more enzymes associated with biosynthesis of the aroma compounds; and isolating the aroma compounds.

101. The method of claim 100, wherein the one or more enzymes is a synthase, a reductase, a transferase or a combination thereof.

102. The method of claim 101, wherein
the synthase is a phenylacetaldehyde synthase, a propenylphenol synthase, an allylphenol synthase, a biologically active fragment or variant thereof or a combination thereof;
the reductase is a phenylacetaldehyde reductase (PAR) wherein the PAR is lycopersicum esculetum phenylacetaldehyde reductase (LePAR1) or a biologically active fragment or variant thereof; and.
the transferase is an acyl transferase (ltCAAT1) or a biologically active fragment or variant thereof.


SEQUENCE LISTING (14 sequences)

SEQ ID NO: 1 - DNA, 25 bp, Artificial Sequence
  RhFCPAAS-F
caccatgggtagcttcccattccac

SEQ ID NO: 2 - DNA, 21 bp, Artificial Sequence
  RhFCPAAS-R
tcaatacgtgctgaggattgc

SEQ ID NO: 3 - DNA, 29 bp, Artificial Sequence
  RhFCPAAS-XHOI-F
caccctcgagatgggtagcttcccattcc

SEQ ID NO: 4 - DNA, 30 bp, Artificial Sequence
  RhFCPAAS-BGLII-R
agatcttcaatacgtgctgaggattgcttg

SEQ ID NO: 5 - DNA, 51 bp, Artificial Sequence
  (RhFCPAAS)-attB5-p35S-F
ggggacaactttgtatacaaaagttgtcgacgaattaattccaatcccaca

SEQ ID NO: 6 - DNA, 74 bp, Artificial Sequence
  (RhFCPAAS)-attB2-tOCS-R
ggggaccactttgtacaagaaagctgggtaagatttaggtgacactatagaatatgcatcactagtaagctagc

SEQ ID NO: 7 - DNA, 25 bp, Artificial Sequence
  LePAR1-F
caccatgagtgtgacagcgaaaaca

SEQ ID NO: 8 - DNA, 21 bp, Artificial Sequence
  LePAR1-R
ttacatagaagatgaacctcc

SEQ ID NO: 9 - DNA, 50 bp, Artificial Sequence
  (LePAR1)-attB1-p35S-F
ggggacaagtttgtacaaaaaagcaggctctagagccaagctgatctcct

SEQ ID NO: 10 - DNA, 48 bp, Artificial Sequence
  (LePAR1)-attB5r-t35S-R
ggggacaacttttgtatacaaagttgtctagagggcccgacgtcgcat

SEQ ID NO: 11 - DNA, 20 bp, Artificial Sequence
  LePAR1-QPCR-F
tgtgaaggcttctgttcgtg

SEQ ID NO: 12 - DNA, 20 bp, Artificial Sequence
  LePAR1-QPCR-R
gcacatgacccgagaagatt

SEQ ID NO: 13 - DNA, 20 bp, Artificial Sequence
  PtaACTIN-QPCR-F
ggtcctcttccaaccttcaa

SEQ ID NO: 14 - DNA, 21 bp, Artificial Sequence
  PtaACTIN-QPCR-R
tcctgggaacatagttgaacc